(19) United States
(12) Patent Application Publication
(10) Pub. No.: US 2003/0149347 A1
(43) Pub. Date: Aug. 7, 2003

(54) Method of evaluating the region around the eye and apparatus for implementing such a method

(75) Inventors: Myriam Kauffmann, Lyon (FR); Roland Bazin, Bievres (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/326,372

(22) Filed: Dec. 23, 2002

(30) Foreign Application Priority Data
Dec. 21, 2001 (FR) 01 16787

(51) Int. Cl. A61B 5/00

(52) U.S. Cl. 600/310

(57) ABSTRACT

A method evaluating the typology of the region extending around the human eye and an apparatus that can perform the evaluation are provided. At least one data item is acquired concerning at least one zone of the region around the eye, with the data item being representative of a pigment present in the blood stream and/or a pigment outside the blood stream. In addition, at least one data item is acquired relating to the relief of at least one zone of the region and/or at least one mechanical property of at least one zone of the region. Based upon the acquired data, information relating to the typology of the region is provided.

### CROSS REFERENCE TO RELATED APPLICATION

This document claims priority to French application number 01 16787 filed Dec. 21, 2001 the entire content of which is hereby incorporated by reference.

1. Field of the Invention

The present invention relates to methods and apparatuses for evaluating the region around the eyes.

### BACKGROUND OF THE INVENTION

2. Discussion of Background

The article entitled “Study of causal factor of dark circles around the eyes”, published in IFSCC Magazine, Vol. 4, No. 4/2001 describes the use of a Doppler laser and of a color meter for taking measurements in the region around the eye and attempting to explain the cause of rings around the eye. However, heretofore, a method and apparatus to easily and/or precisely obtain measurements/data to evaluate the eye region has not been available, particularly in an automated fashion. Accordingly, a need exists for a method and apparatus to evaluate the typology of the region that extends around the human eye, in particular concerning rings, bags, wrinkles in the corner of the eye, and irregularities of the skin and of the eyelids.

### SUMMARY OF THE INVENTION

In accordance with one of the advantageous aspects of the invention, a method is provided for facilitating evaluation of the typology of the skin, and particularly the region around the eye. The method includes enabling at least one data item to be acquired concerning at least one zone of the region, with the data item being associated with at least one pigment present in the blood stream and/or at least one pigment outside the blood stream. The method further includes enabling at least one data item to be acquired relating to the relief of at least one zone of the region and/or at least one data item to be acquired relating to at least one mechanical property of at least one zone of the region. Based upon the previously-acquired data, at least one piece of information relating to the typology of the region is delivered.

The pigment present in the blood stream (all types of blood vessels) is hemoglobin, in its oxidized form or its non-oxidized form, for example. The pigment present outside the blood stream is, for example: a melanin, bilirubin, or hemosiderin. With the invention, it is possible to more easily and/or more precisely evaluate the typology of the region extending around the human eye, and the invention makes it possible in particular, to automate this evaluation, at least in part. The invention allows the region around the eye of a person to be classified into various categories and, as a function of such classification, to determine the treatment that might be applied, such as a particular non-therapeutic treatment. For example, the invention can make it possible to avoid prescription errors such as administering a vascular care product to a person whose rings are melanic, where the product will have no effect on such rings because their color is due to the natural coloring of the skin.

At least one data item associated with at least one pigment present in the blood stream and/or with at least one pigment outside the blood stream may be acquired by acquiring at least one data item concerning color. In a variant, or additionally, it may be acquired by acquiring data concerning at least one temperature measurement and/or at least one speed measurement of blood circulation.

According to a further aspect of the invention, the method can include acquiring at least one data item relating to the circumstances surrounding the advent and/or the development of at least one characteristic of the skin region over time. For example, this data may be the result of taking measurements at different times or of obtaining answers to a questionnaire. For example, a questionnaire can include questions relating to the circumstances associated with the arrival of rings or bags, and with their reversibility. The questionnaire may also have questions concerning the existence of any discomfort, for example, a pricking sensation which can be associated with an allergic event.

In accordance with another aspect of the invention, the method includes acquiring at least one data item relating to the color of at least one zone of the region, and acquiring at least one data item relating to the relief of at least one zone of the region. In addition, at least one data item relating to at least one mechanical property of at least one zone of the region is acquired, and on the basis of the previously-acquired data items, at least one piece of information relating to the typology of the region is delivered. The acquired data can be compared with predetermined data, and at least one piece of information concerning the typology of the region can be issued in the light of or in response to the result of the comparison.

According to another aspect of the invention, the method can include the step of determining the transparency of the skin in at least one zone of the region. The ease with which light passes through the skin can be responsible, at least in part, for rings being visible, since the rings might be due to the presence of underlying blood vessels.

By way of example, in order to acquire at least one data item concerning the color of at least one zone of the region, at least one of the following can be used: a video camera, a scanner, a digital camera, a spectrocolorimeter, a calorimeter, a color chart, a SCIASCOPE® (as described in WO 00/75637, which is incorporated herein by reference), a MEXAMETRE® (as sold by the supplier Courage et Khazaka), or a thermal camera. A SCIASCOPE is an apparatus that includes a light source for projecting light to illuminate an area of a tissue sample, a photoreceptor for receiving light re-emitted by the illuminated area of tissue, and a spectroscopic analyzer for monitoring the re-emitted light. In addition, a comparator is provided for comparing variations in the intensity and spectral characteristics of the re-emitted light with respect to the intensity and spectral characteristics of the projected light at different wavelengths, and a record of the intensity and spectral characteristics of light re-emitted by a reference sample of such tissue is provided, and a control signal is emitted in response to any such variations. A MEXAMETRE is an apparatus which obtains measurements based on absorption. With such a device, probe emits light of three defined wavelengths. The melanin is measured by two wavelengths which have been chosen in order to achieve different absorption rates by the melanin pigments. For an erythema measurement, two different wavelengths are used to measure the absorption capacity of the skin. One of these wavelengths corresponds to the spectral absorption peak or hemoglobin. The other wavelength is chosen to avoid other color influences (e.g. bilirubin). A receiver measures the light reflected by the skin. The positions of emitter and receiver guarantee that only diffuse and scattered light is measured. As the quantity of emitted light is defined, the quantity of light absorbed by the skin can be calculated. It is to be understood that, in accordance with the invention, while acquiring the at least one data item concerning the color of at least one zone of the region, the region can be illuminated using visible, ultraviolet (UV), or infrared light. It is also possible to acquire data concerning the color of at least one zone of the region by successively illuminating the zone with different kinds of lighting.

By way of example, it is possible to acquire at least one data item relating to the relief of at least one zone of the region by one of the following: a system for projecting fringes; a stereoscopic viewing system; an ultrasound imaging system; or a slit lamp. Also, by way of example, it is possible to acquire at least one data item relating to the mechanical properties of at least one zone of the region by one of the following: a mechanical feeler or probe device; a pinching device; a twisting device; a suction device; a device for projecting a fluid (liquid or a gas); a tonometer; a plethysmographic device.

In accordance with an aspect of the invention, it is possible in a first time interval to perform at least a first acquisition of at least one data item relating to the color of at least one zone of the region and/or of at least one data item relating to the relief of at least one zone of the region and/or of at least one data item relating to at least one mechanical property of at least one zone of the region. It is then possible in a second interval of time following the first to proceed with at least one second acquisition of at least one data item relating to the color of at least one zone of the region and/or of at least one data item relating to the relief of at least one zone of the region and/or of at least one data item relating to at least one mechanical property of at least one zone of the region. In particular, when the zones examined during the first and second acquisitions are the same zone, it is possible to compare the results of the first and second acquisitions and to deduce from the results of the comparisons at least one piece of information relating to the typology of the region.

The information obtained on the basis of the various acquisitions performed may be information relating to rings, and in particular information enabling rings to be categorized into a set of categories. By way of example, such a set can include three types of rings, where the various types of rings may be, for example: ethnic rings; false rings; and rings that are at least partially reversible.

The information relating to the typology of the region around the eye can also be information relating to bags, for example information enabling bags to be categorized in a set of categories. One such set can include, for example, four categories: hollow bags due to sagging tissue; full bags due to chambers of protruding fat; full bags due to ptosis of tissue (e.g. sagging of elevator muscles); and full bags due to lymphedema swelling.

The information relating to the typology of the region around the eye may also be information relating to wrinkles in the corner of the eye, for example, their number, their length, and their depth.

In accordance with another aspect of the invention, a method is provided for evaluating the effect on the appearance of the region around the eye, of applying a substance, in particular a cosmetic, or determining the effectiveness of other treatments. The method can include, prior to application of the substance and/or treatment (such as a cosmetic or surgical treatment) acquiring data associated with at least one pigment present in and/or outside the blood stream, in particular at least one data item concerning color, and data concerning the relief and/or mechanical properties of at least one zone of the region around the eye. The substance is then applied and/or the treatment is performed. Thereafter, data is acquired that is associated with the at least one pigment present in and/or outside the blood stream, in particular at least one data item concerning color, and data is acquired concerning relief and/or mechanical properties of at least one zone of the region around the eye. The results of the first and second (“before” and “after”) acquisitions are then compared and the incidence of the application of the substance or the effectiveness of the treatment on the appearance of the region around the eye is determined.

To understand what is meant by “cosmetic”, reference can be made to EEC Directive No. 76/768 as modified by EEC Directive No. 96/35 of Jun. 14, 1993. Cosmetic treatment means any non-therapeutic treatment by use of a cosmetic as defined above.

In accordance with another aspect of the present invention, an apparatus is provided which can acquire at least one data item associated with at least one pigment present in the blood stream and/or at least one pigment outside the blood stream, in particular an item relating to the color of at least one zone of the region. In addition, the apparatus can acquire at least one data item relating to the relief of at least one zone of the region, and/or at least one data item relating to at least one mechanical property of at least one zone of the region.

The term “apparatus” is used herein for both a set of devices and also a device of unitary construction, possibly suitable for being held in one hand. In other words, various components of the apparatus can be housed separately or the apparatus can be a single unit. It is also to be understood that various different combinations of features can be provided in a given apparatus according to the invention.

In accordance with a further aspect of the invention, the apparatus can include a treatment arrangement for comparing the acquired data with stored data and for generating at least one piece of information concerning the typology of the observed region as a function of the result of the comparison. The generated information can be a numerical value or it can be information in non-numerical form. The apparatus can be arranged to enable data to be acquired relating to the variation over time of at least one characteristic of the region in question.

The apparatus can include a colorimeter acquisition arrangement having at least one of the following: a video camera; a scanner; a digital camera; a spectrocolorimeter; a calorimeter; a color chart; and a thermal camera. The device can also include lighting to enable the region to be illuminated with visible, ultraviolet, or infrared light. The apparatus can also include an arrangement to enable the colorimetric acquisition to be performed under different lighting or by transparency.

The apparatus can also acquire a relief, with at least one of the following: a system for projecting fringes; a stereoscopic viewing system; an ultrasound imaging system; and a slit lamp.

The apparatus can include an arrangement for acquiring at least one mechanical property, with at least one of the following: a mechanical feeler or probe device; a pinching device; a twisting device; a suction device; a device for projecting liquid or gas; a tonometer device; and a plethysmographic device.

The apparatus can also store data acquired at different instants with time elapsing between collection of data so that changes over time can be determined or the data taken at different times can be compared.

The apparatus can also include a mask arranged to have its entire outline pressed against a region around the eye.

The region around the eye may be evaluated for a non-therapeutic purpose, in particular for an esthetic purpose.

In accordance with another aspect, the invention also provides a method of diagnosing at least one state of the region around the eye. The method includes acquiring at least one data item concerning at least one zone of the region, with the data item being associated with at least one pigment present in the blood stream and/or at least one pigment outside the blood stream. In addition, at least one data item is acquired relating to the relief of at least one zone of the region and/or at least one data item relating to at least one mechanical property of at least one zone of the region. Then, a diagnosis is delivered, such as a non-medical diagnosis, in response to the previously acquired data, i.e., at least on the basis of the previously-acquired data.

In accordance with another aspect, the invention also provides a method of prescribing a treatment, in particular, a cosmetic treatment. The method includes acquiring at least one data item concerning at least one zone of the region, the data item being associated with at least one pigment present in the blood stream and/or at least one pigment outside the blood stream. In addition, at least one data item is acquired relating to the relief of at least one zone of the region, and at least one data item is acquired relating to at least one mechanical property of at least one zone of the region. A treatment is then prescribed in response to the acquired data, i.e., at least on the basis of the previously-acquired data. The prescribed treatment can include a substance having one or more of a depigmenting active agent (e.g. kojic acid); a UV filter; an anti-allergy substance; and an anti-inflammatory substance.

In accordance with another aspect, the invention also provides a kit enabling an individual to perform a self-diagnosis. The kit can include an arrangement (e.g. a mirror) to enable at least one data item to be acquired concerning at least one zone of the region, with the data item being associated with at least one pigment present in the blood stream and/or at least one pigment outside the blood stream. The kit can further include an arrangement or accessory enabling at least one data item to be acquired relating to the relief of at least one zone of the region and/or at least one data item to be acquired relating to at least one mechanical property of at least one zone of the region. The kit further enables (e.g., with an atlas) a diagnosis to be delivered, in particular a non-medical diagnosis, at least on the basis of the previously-acquired data.

In accordance with yet another aspect, the invention also provides a method of manufacturing a product, in particular a cosmetic. The method includes acquiring at least one data item concerning at least one zone of the region, with the data item being associated with at least one pigment present in the blood stream and/or at least one pigment outside the blood stream. In addition, at least one data item can be acquired relating to the relief of at least one zone of the region and/or at least one data item relating to at least one mechanical property of at least one zone of the region. A product, in particular a cosmetic, is then prepared at least on the basis of the previously-acquired data.

In accordance with the invention a method of generating a panel of potential users of a product, in particular a cosmetic, is also provided. The method includes, for each individual in a group, acquiring at least one data item concerning at least one zone of the region, with the data item being associated with at least one pigment present in the blood stream and/or at least one pigment outside the blood stream. In addition, at least one data item is acquired relating to the relief of at least one zone of the region and/or at least one data item relating to at least one mechanical property of at least one zone of the region. A selecting step is then performed to select individuals of the group for which the previously-acquired data satisfies at least one predetermined criterion. The method can further include:

i) generating a panel;

ii) preparing a product, in particular a cosmetic;

iii) applying or administering the product to individuals of the panel;

iv) performing a new evaluation:

v) comparing the results of the new evaluation with those of the preceding evaluation(s), in order to determine the effectiveness of the product; and

vi) if the effectiveness of the product is deemed to be insufficient, modifying the formulation of the product and/or its dosage. Steps iii), iv), and v) can also be repeated until effectiveness is obtained that is deemed to be satisfactory.

In accordance with yet another aspect, the invention also provides a method of determining the effectiveness of a treatment, in particular a cosmetic treatment. The method includes performing a first evaluation of the region around the eye, and then performing a treatment to the region. Then, a new evaluation is performed, and the results of the evaluations are compared to deduce useful information concerning the effectiveness of the treatment.

### BRIEF DESCRIPTION OF THE DRAWINGS

The invention will be better understood on reading the following description of non-limiting implementations and on examining the accompanying drawings, which form an integral portion of the description and in which:

FIG. 1 is a schematic illustration of an example of apparatus that may be used for evaluating the region around the eye;

FIG. 2 is a schematic illustration on a larger scale of the FIG. 1 apparatus, showing the various component elements of the apparatus;

FIG. 3 shows an example of an apparatus suitable for use for measuring the color and/or the relief of the region around the eye;

FIG. 4 illustrates the face positioned in the opening of the sphere of the FIG. 3 apparatus;

FIG. 5 depicts an example of the location of melanic rings;

FIG. 6 depicts an example of the location of rings other than melanic;

FIG. 7 depicts an example of the location of a bag;

FIG. 8 depicts an example of determining a characteristic of the typology of the region around the eye utilizing a sequence of images;

FIG. 9 is a block diagram showing an aspect of an implementation of the method of the invention;

FIG. 10 schematically represents data being transmitted over a computer network, for example, in order to evaluate the acquired data remotely; and FIG. 11 illustrates an example of apparatus enabling a product to be prepared as a function of the result of evaluating the region around the eye.

### DETAILED DESCRIPTION OF THE PREFERRED EMBODIMENTS

The region around the eye may be evaluated in accordance with the invention by an apparatus of unitary construction or by apparatus constituted by a set of separate devices. The evaluation can be performed while the eye is open, closed, or where the eye is successively closed and then open or vice versa. Evaluation may also be performed on the face while it is stationary and relaxed or smiling and contracted.

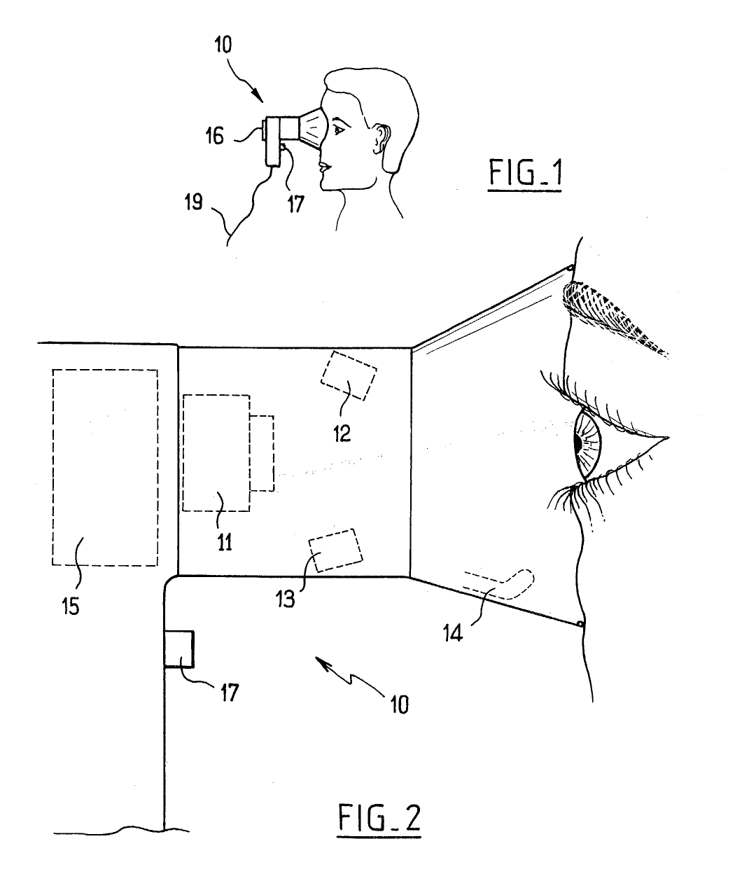

As an example of a unitary construction, FIGS. 1 and 2 show an apparatus 10 combining various elements enabling data to be acquired relating, for example, to the color or the transparency, to the relief, and/or to the mechanical properties of at least one zone of the region around the eye.

The apparatus 10 can include, for example, a camera 11, and at least one lighting device 12.

By way of example, the camera 11 is of the charge-coupled device (CCD) type and the acquired image can be processed in order to determine the color of each pixel and to define zones that have substantially the same color, in order, for example, to evaluate the extent and/or the locations of rings due to one or more predetermined pigments, for example. The outlines of rings due to pigments that circulate with the blood may be determined, for example, by detecting zones of greater redness in the image acquired by means of the camera 11. The camera may previously be calibrated by placing a reference scale of colors in its field of view.

The apparatus 10 may also include, for example, at least one device 13 for evaluating relief, enabling data concerning relief to be acquired for at least one zone of the region around the eye, for example a device for projecting fringes. For example, by projecting fringes and using a camera, the shape of the fringes can be identified to determine the relief based upon the shape of the projected fringes. For example, fringes would be parallel for a planar surface and non-parallel for a non-planar surface. It is also possible to use devices other than a fringe-projecting device to acquire data concerning relief. For example, it is possible to use a stereoscopic viewing device or an ultrasound telemeter device, this list not being exhaustive or limiting.

The apparatus 10 may also include at least one mechanical evaluation device 14 possibly suitable for acting in co-operation with the camera 11 and the device 13 for evaluating relief, in order to determine at least one mechanical property of at least one zone of the region around the eye. By way of example, the mechanical evaluation device 14 can include a nozzle for projecting a jet of compressed air against at least one zone of the region around the eye in order to deform the zone, e.g., the bottom eyelid or the top of the cheek bone. A mechanical property can thereby be evaluated, e.g., based on the deformation resulting from the jet of compressed air.

By way of example, the lighting device 12 can include at least one light source such as an incandescent lamp or a discharge lamp or a light-emitting diode (LED) or a laser diode, or a combination of these various sources. The lighting device 12 could also include at least one light source associated with a light-diffusing system so as to obtain substantially uniform illumination of the region around the eye. In particular, the lighting device 12 can include a plurality of light sources distributed circumferentially, for example. The lighting device 12 could also include one or more light guides, in particular optical fibers. Further, the lighting device 12 can be arranged to illuminate the region around the eye with light that presents well-defined spectral characteristics, e.g., selected to make a particular pigment of the skin show up as much as possible, for example, ferritin or oxidized hemoglobin.

The lighting device 12 could also include a lighting arrangement to illuminate the skin successively with different wavelengths. Thus, the skin can be illuminated successively with visible light, with infrared light, and/or with ultraviolet light, for example.

The lighting device 12 can also be arranged, and in particular pointed, in such a manner as to enable the shadow effect of bags to be eliminated for a certain type of bag. By measuring the degree of attenuation of the shadow effect under this particular lighting, it is possible to deduce information concerning the extent to which the bags project.

For acquiring data concerning mechanical properties, it is also possible to use expedients other than a device for projecting compressed air for providing other forms of mechanical interaction with the skin with non-limiting examples including devices operating by feeling/probing, pinching, twisting, or suction.

The device for evaluating relief can be arranged to co-operate with the mechanical evaluation device, for example in order to measure the way in which relief varies in response to mechanical stress. Thus, for example, the zone under study can be stressed mechanically and the resulting variation of its relief can be measured optically in order to determine the firmness and/or the elasticity of the skin in this zone.

The apparatus 10 can include a processor 15 for processing the signals issued by the various elements for evaluating color, transparency, relief, and/or mechanical properties, with such an arrangement including, e.g., one or more microprocessors or other components suitable for performing the necessary data processing operations. Further, the processing of data could be performed within the device as a stand alone system, or the apparatus could provide signals or data to an external processor, such as a personal computer. Further, as discussed below, data could be transmitted to a remote location, e.g., via the Internet, where processing and/or a recommended treatment is provided.

The apparatus 10 can also include a display 16, e.g., on its side opposite from the opening which is placed around the eye, together with at least one control button 17 enabling its operation to be controlled while acquiring data.

The display 16 can display digital or other information relating to the observed region, for example, the type of rings or bags. In addition, the apparatus 10 can be powered entirely independently, or it can be connected to an electrical power supply.

In the example described, the apparatus 10 includes a mask 18 of substantially frustoconical shape arranged to have its entire perimeter pressed against the face around the eye, matching the shape of the face or a portion of the face, for example the shape of the eyebrow and a portion of the nose. It is to be understood, however, that other shapes for the mask are also possible.

Prior to being pressed against the face, the outline of the mask can lie completely in a plane, in which case the mask can include a gasket of elastically-deformable material for defining its outline. When the mask is pressed against the face around the eye, deformation of the gasket then enables it to match the shape of the face. Alternately, the mask need not have a gasket and can itself be made of a material that is elastically deformable so as to be capable of matching the shape of the face. Further, the outline of the mask need not lie in a plane, but may be of a shape suitable for enabling it, without deforming or with little deformation, to match the relief of the face in the region around the eye.

As noted above, the apparatus 10 can omit an arrangement for processing the information delivered by the various elements used for acquiring data. By way of example, the data can be processed by a personal computer or some other equipment external to the apparatus 10, or the data processing can be performed both by the apparatus used for acquiring the data and by data processing equipment or a system to which the acquisition apparatus is connected and with which it can exchange information by means of a cable or a wireless link, for example.

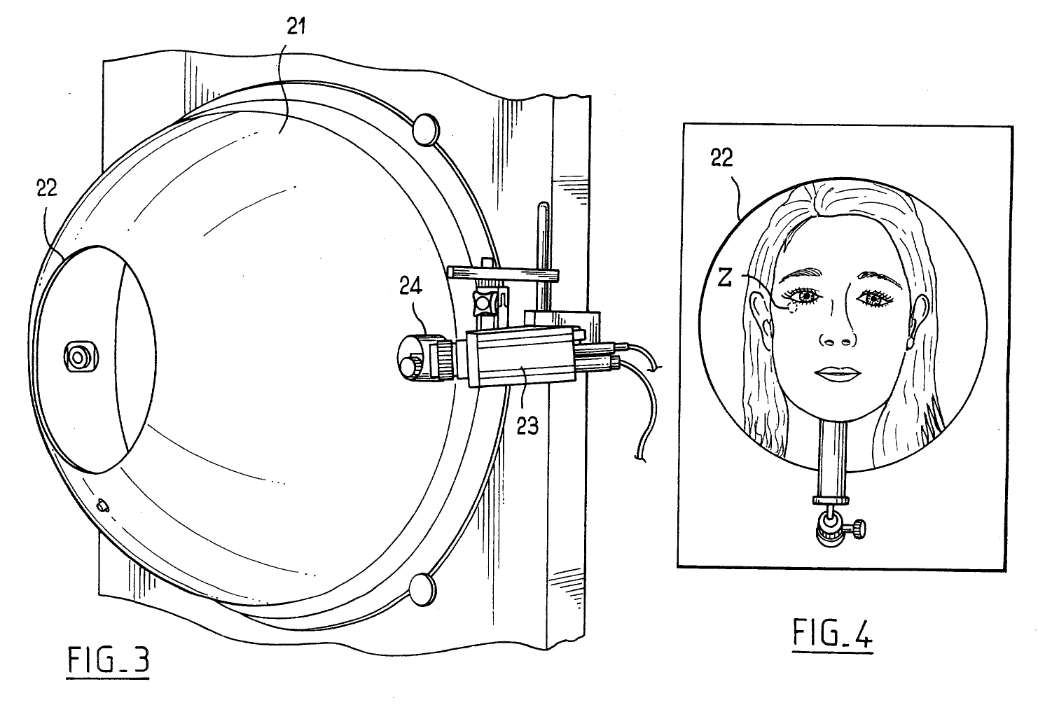

FIGS. 3 and 4 illustrate an example of an apparatus 20 enabling colorimetric data to be acquired and enabling the relief of the region around the eye to be observed. The apparatus 20 is not of unitary construction, since it includes a set of devices capable of being used independently from one another, rather than simultaneously, with the devices or component capable of being moved relative to one another.

In the example shown, the apparatus 20 includes a sphere 21 (more precisely a section of a sphere) having an opening 22 in which a person can place the face in order to enable the region around the eye to be evaluated, and a plurality of cameras 23 enabling at least one zone of the region around the eye to be observed, for example the zone Z defined by a broken-line circle in FIG. 4. There are three cameras 23 in the example of FIG. 3. Two of the cameras are positioned to observe images through openings on the lateral sides of the sphere 21, while the third (not shown) can observe images through the opening 22, thereby enabling the face to be observed from in front and from the side through openings 24 in the sphere 21. Although three cameras are provided in the illustrated embodiment, it is to be understood that the number of cameras can be varied.

The various cameras 23 can be used not only to acquire information relating to the color of the region around the eye, but also to its relief, by stereoscopy.

Other devices can also be used, for example that described in application WO 00/75637, the content of which is incorporated herein by reference.

In an aspect of the invention, the data acquisition arrangement can be used:

to acquire at least one data item relating to the color of at least one zone of the region around the eye;

to acquire at least one data item relating to the relief of at least one zone of the region and/or at least one data item relating to mechanical properties of at least one zone of the region; and

at least on the basis of the previously-acquired data, to deliver at least one piece of information relating to the typology of the region.

The invention can be particularly advantageously utilized for evaluating regions around the eye, and more particularly for evaluating rings or bags around the eyes.

Rings may have several causes. It is possible in accordance with the invention to distinguish between at least three types of rings, namely: so-called “ethnic” or “melanic” rings due to the natural pigmentation of the skin; false rings, i.e. rings due to a light and shadow effect because of the relief in the region around the eye; and “vascular” rings associated for example with illness or a lack of sleep. These various categories of ring are listed in Table I below.

TABLE I

| Type of ring | Color | Location | Origin | Variation |
|---|---|---|---|---|
| Vascular | Violet, blue, | Bottom eyelid, | Blood | More or less |
| | red | corner with the | visible by | reversible |
| | | top para-nasal | trans- | |
| | | zone | parency | |
| Ethnic | Brown, gray | Bottom and | Melanins | Not reversible |
| | | top eyelids | | (darkens with |
| | | | | exposure to the |
| | | | | sun) |
| False rings | — | Bottom eyelid | Light and | Depends on |
| | | | shadow | variation of |
| | | | effect | bags |

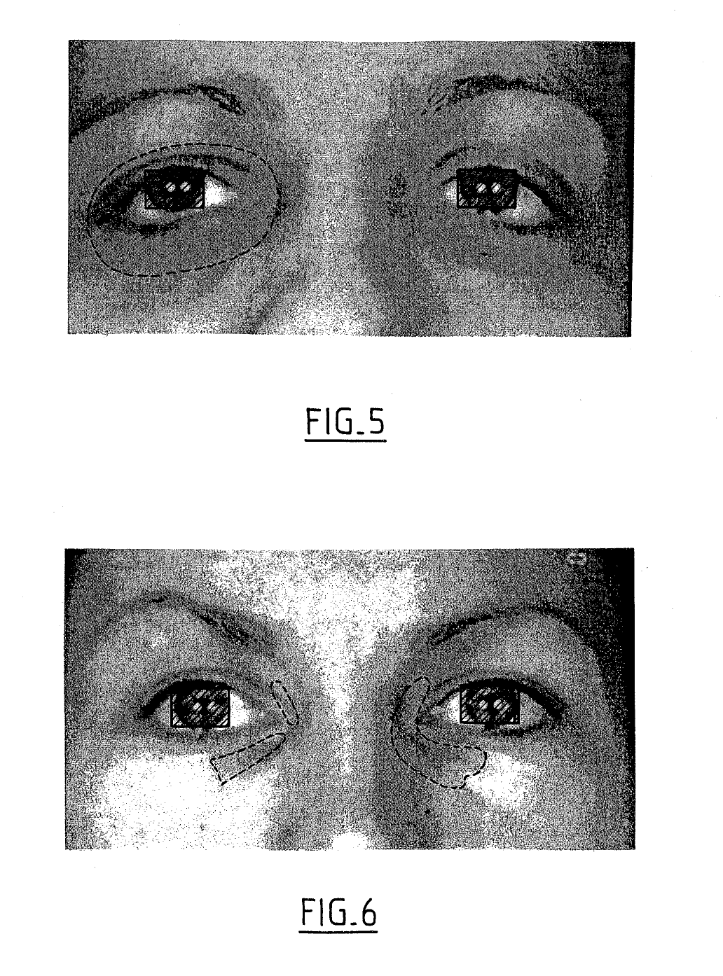

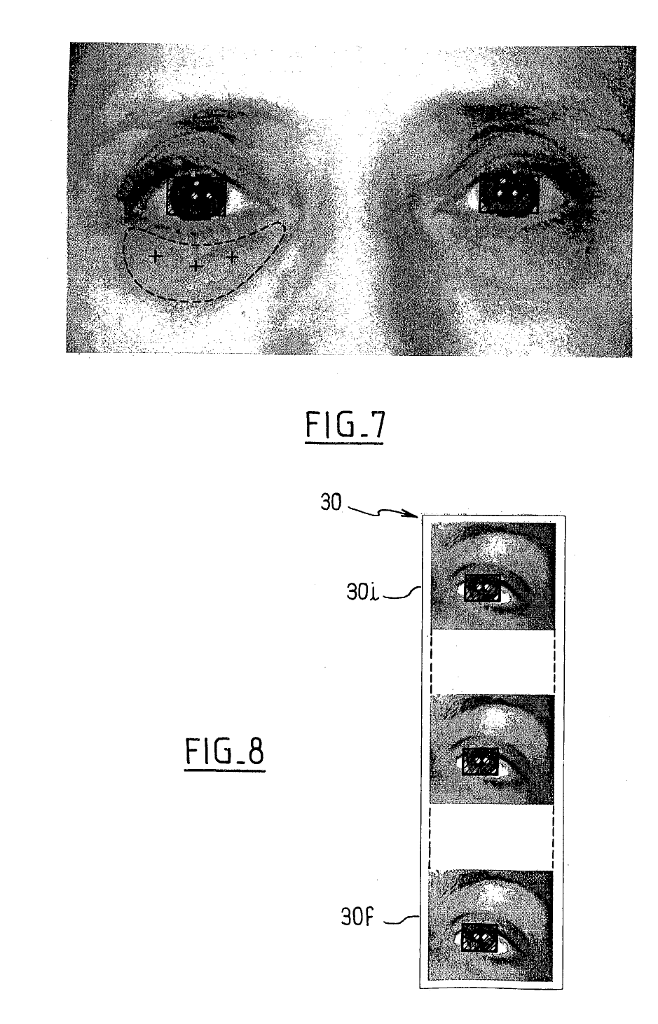

Dashed or broken lines are used in FIG. 5 to show an example of a location of an ethnic ring, in FIG. 6 to show an example of the location of vascular rings, and in FIG. 7 to show an example of the location of false rings.

The colorimetry, relief, and/or mechanical property data can be compared with predetermined data, for example, data stored in a memory, in order to determine the types of rings. For example, a comparison can be performed automatically or otherwise between an image acquired using a camera and a sequence of images within which at least one characteristic of the typology of the region around the eye varies, for example, with a continuous progression as shown in FIG. 8. In FIG. 8, there is shown a sequence of images 30 including an initial image 30i and a final image 30f, with such a sequence being generated by a morphing program, for example. By way of example, the acquired image relating to the region around the eye and serving as a reference may be displayed on a screen simultaneously with an image of the sequence so as to enable an operator or a person performing self-diagnosis to select the image in the sequence which in their opinion is the closest to the reference image. Comparing the reference image and the images in the sequence can also be performed without displaying the images of the sequence, for example by means of an image recognition engine. Under such circumstances, it can be particularly advantageous to define automatically the outlines of rings by analyzing the color at each point of the image. An atlas comprising a plurality of images representing different types of rings or bags may also be used so that the image of the subject can be compared with those in the atlas.

Acquiring the relief of the region around the eye, possibly in combination with acquiring mechanical characteristics, makes it possible to determine the type of a bag.

Relief can be acquired at a plurality of points, for example with the points marked by crosses in FIG. 7, in order to determine whether the surface of the skin is generally concave or convex, respectively representative of a bag that is hollow or full.

Hollow bags are essentially due to morpho-anatomic causes, to tissue becoming finer or sagging, whereas full bags are of congenital origin, or may be of physiological origin, for example due to inflammation.

Table II below lists the various types of bags.

TABLE II

| Type | Mechanical | | | |
|---|---|---|---|---|
| of bag | characteristics | Location | Origin | Variation |
| Hollow | Stretched skin | Bottom | Tissue sagging | Not reversible |
| | | and | with age | |
| | | top | | |
| Full, not | Non specific | Bottom | Protrusion of | Not reversible |
| reversible | | | chambers of | |
| | | | fat | |
| Full and | Non specific | Top | Ptosis | More or less |
| partially | | | | reversible |
| reversible | | | | |
| Full and | Stretched skin | Bottom | Lymphedema | Reversible |
| reversible | | and | swelling | |
| | | top | | |

Bag type may optionally be determined using a morphing program, on the same lines as determining ring type, to provide plural images or data for comparison with the acquired data of the subject.

Data concerning the color, the mechanical properties, and relief may be associated with acquiring data from a questionnaire. By way of example, such a questionnaire can be displayed on a computer screen and can include questions relating to how the rings or the bags have varied over the course of a day, and to the existence of positive and negative contributory factors such as medical treatment or a particular state of mind, exposure to certain environments, or wearing sunglasses while exposed to the sun, for example. The questionnaire can be used in particular to acquire data correlating rings or bags or rings with events, e.g., concerning the presence or absence of rings or bags first thing in the morning or in the evening, and more generally on the reversible or non-reversible (or more chronic) nature of rings or bags, according to the person being examined.

According to one aspect, by way of example, a substance can optionally be administered in order to generate tears. For example, the eye may be exposed to the substances given off by an onion peel or other substance to generate tears. By comparing the appearance of the region around the eye before and after exposure to onion peel, it is easier to determine certain characteristics, for example, to measure the extent to which bags swell.

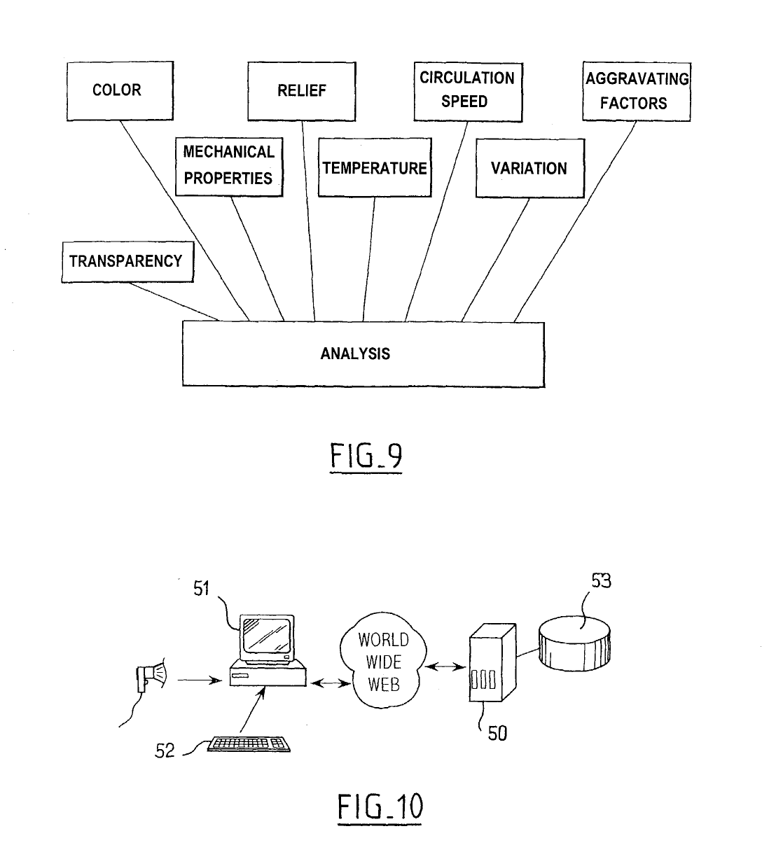

The various data items may be analyzed, as shown in FIG. 9, so as to classify rings and bags in the various above-specified categories. It is to be understood that the specific data items or combinations of data items can be varied depending upon the level of sophistication desired. For example, a large number of data items can be input and analyzed for a higher level system. However, a less sophisticated system could yield advantageous information based on one, two, or a small number of data items while being more economically modest. Accordingly, it is to be understood that the specific set of data items that could be utilized in a given apparatus or method can vary.

Depending on the results of the analysis, a cosmetic, pharmacological, or surgical treatment may be proposed.

With ethnic rings, depigmentation can be proposed, for example, and with false rings, surgical treatment can be performed to flatten the bags. Hollow bags can be filled and full bags due to protrusion of chambers of fat can be treated by removing some of the fat. Full bags due to ptosis can be treated by a face lift, by injecting or applying myorelaxing agents or myostimulating agents to the lowering or raising muscles or to isolated muscular fibers.

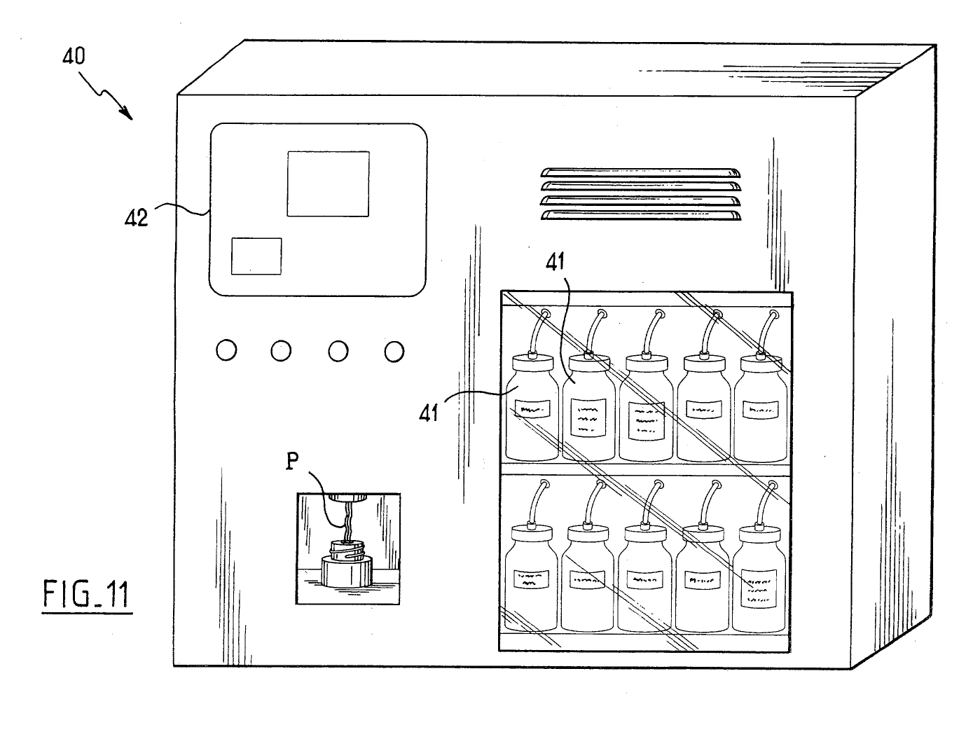

The results of acquiring colorimetry, relief, and/or mechanical data, e.g. using the above-described apparatus 10, possibly together with other information as input for example by means of a keyboard 52 connected to a personal computer 51 can be transmitted remotely, e.g., over the Internet, to a server 50 containing or connected to a database 53, as shown in FIG. 10. The server 50 may be programmed, in response to the transmitted data, to diagnose and recommend care or makeup and to give advice. The server 50 may also be arranged to enable various data items to be collected in order to constitute a multi-vectorial data bank, for example, in which each vector is specific to an individual and includes amongst other components: that individual's bag type and ring type.

Thus, for example, it is possible as a function of geographical zone to determine which type or types of bags and rings predominate and then to sell suitable products in that geographical zone.

The means for acquiring data relating to color, transparency, relief, and/or mechanical properties of the region around the eye may be connected to apparatus 40 as shown diagrammatically in FIG. 11 suitable for preparing a cosmetic or treatment product P as a function of the result of the evaluation and using a plurality of receptacles 41 containing different ingredients.

The apparatus 40 can include, for example, a screen 42, in particular a touch-sensitive screen, enabling a user to enter replies to a questionnaire.

The apparatus 40 may be present in an institute or at a point of sale, for example, and it may enable a personalized makeup and/or care product to be provided corresponding as closely as possible to the bag and ring types of a previously-examined person.

Obviously, numerous modifications and variations of the present invention are possible in light of the above teachings. It is therefore to be understood that, within the scope of the appended claims, the invention may be practiced otherwise than as specifically described herein.

What is claimed is:

1. A method of evaluating the typology of the region extending around the human eye, the method comprising:
enabling acquisition of at least one data item concerning at least one zone of said region, the data item being associated with at least one of a pigment present in the blood stream and a pigment outside the blood stream;
enabling acquisition of at least one data item relating to at least one of the relief of at least one zone of said region and at least one mechanical property of at least one zone of said region; and
at least on the basis of the previously-acquired data, delivering at least one piece of information relating to the typology of said region.

2. A method according to claim 1, wherein the acquisition of at least one data item associated with at least one of a pigment present in the blood stream and a pigment outside the blood stream comprises acquiring at least one data item relating to the color of at least one zone of said region.

3. A method according to claim 1, further comprising:
acquiring at least one data item relating to circumstances under which at least one characteristic of said region appeared.

4. A method according to claim 1, including the step of determining transparency of at least one zone of said region.

5. A method according to claim 1, comprising:
acquiring at least one data item relating to the color of at least one zone of said region;
acquiring at least one data item relating to a relief of at least one zone of said region;
acquiring at least one data item relating to at least one mechanical property of at least one zone of said region; and
at least on the basis of the previously-acquired data items, delivering at least one piece of information relating to the typology of said region.

6. A method according to claim 1, wherein the acquired data is compared with predetermined data and wherein at least one piece of information relating to the typology of said region is delivered in the response to the result of the comparison.

7. A method according to claim 1, wherein acquiring of at least one data item relating to the color of at least one zone of said region, includes utilizing at least one of the following to acquire at least one data item relating to color: a video camera; a scanner; a digital camera; a colorimeter; a spectrocolorimeter; a color chart; a device including a light source for projecting light to illuminate an area of tissue, a photoreceptor for receiving light re-emitted from the illuminated area and a spectroscopic analyzer; an apparatus that measures color based upon absorption of light; and a thermal camera.

8. A method according to claim 1, further including while acquiring said at least one data item relating to the color of at least one zone of said region, illuminating said region with visible light.

9. A method according to claim 1, further including while acquiring said at least one data item relating to the color of at least one zone of said region, illuminating said region with ultraviolet light.

10. A method according to claim 1, further including while acquiring said at least one data item relating to the color of at least one zone of said region illuminating, said region with infrared light.

11. A method according to claim 1, wherein data concerning the color of at least one zone of said region is acquired by illuminating said zone in succession with different kinds of lighting.

12. A method according to claim 1, wherein at least one data item relating to the relief of at least one zone of said region is acquired utilizing at least one of the following: a system for projecting fringes; a stereoscopic viewing system; an ultrasound imaging system; and a slit lamp.

13. A method according to claim 1, wherein at least one data item relating to the mechanical properties of at least one zone of said region is acquired utilizing at least one of the following: a mechanical feeler; a pinching device; a twisting device; a suction device; a device for projecting liquid or gas; a tonometer; and a plethysmographic device.

14. A method according to claim 1, including:
performing, in a first time interval, at least a first acquisition of at least one data item relating to at least one of: (a) the color of at least one zone of said region, (b) the relief of at least one zone of said region, and (c) a mechanical property of at least one zone of said region;
performing in a second time interval following the first time interval, at least one second acquisition of at least one data item relating to at least one of: (a) the color of at least one zone of said region, (b) the relief of at least one zone of said region, and (c) a mechanical property of at least one zone of said region;
comparing the results of the first and second acquisitions; and
identifying at least one piece of information relating to typology of said region from the results of the comparing of the first and second acquisitions.

15. A method according to claim 1, wherein said at least one piece of information relating to typology of said region comprises information relating to rings.

16. A method according to claim 15, wherein said at least one piece of information relating to typology of said region comprises information to classify rings into a category from amongst a set comprising a plurality of categories.

17. A method according to claim 16, wherein said set comprises at least the following categories of ring: ethnic rings; false rings; and vascular rings.

18. A method according to claim 1, wherein said at least one piece of information relating to the typology of said region comprises information relating to bags.

19. A method according to claim 18, wherein said at least one piece of information relating to the typology of said region comprises information to classify bags into a category from a set of a plurality of categories.

20. A method according to claim 19, wherein the plurality of categories include hollow bags due to tissue sagging; full bags due to protrusion of chambers of fat; full bags due to ptosis of certain tissues; full bags due to lymphedema swelling.

21. A method according to claim 1, wherein said at least one piece of information relating to the typology of said region comprises information relating to wrinkles in the corner of the eye.

22. A method according to claim 1, further including acquiring data relating to how at least one characteristic of said region varies over time.

23. A method of evaluating at least one of the incidence on the appearance of the region around the eye of applying a cosmetic and the effectiveness of a cosmetic treatment, comprising:
on a first occasion, acquiring data associated with color of at least one pigment present in and/or outside of the blood stream, and acquiring data relating to at least one of relief and at least one mechanical property of at least one zone of the region around the eye;
after acquiring said data on said first occasion, applying at least one of a cosmetic and a cosmetic treatment;
subsequently, on a second occasion, acquiring data associated with color of said at least one pigment present in and/or outside of the blood stream and acquiring data relating to relief and/or at least one mechanical property of at least one zone of the region around the eye; and
comparing the results of the first and second acquisitions in order to determine the incidence on the appearance of the region around the eye of the application of the cosmetic or of the cosmetic treatment.

24. Apparatus for acquiring data relating to typology of the region extending around the human eye, the apparatus comprising acquisition means for acquiring:
at least one data item associated with at least one of a pigment present in the blood stream and a pigment outside the blood stream; and
at least one data item relating to at least one of a relief of at least one zone of said region and a mechanical property of at least one zone of said region.

25. The apparatus of claim 24, wherein said data item associated with at least one pigment includes data relating to color of at least one zone of said region.

26. The apparatus according to claim 25, further comprising processor means for comparing acquired data with stored data and for generating at least one piece of information relating to the typology of said region as a function of the comparison.

27. The apparatus according to claim 25, the apparatus being arranged to enable data to be acquired relating to variation of at least one characteristic of said region over time.

28. The apparatus according to claim 25, including colorimetry acquisition means comprising at least one a video camera; a scanner; a digital camera; a colorimeter; a spectrocolorimeter; a color chart; a device including a light source for projecting light to illuminate an area of tissue, a photoreceptor for receiving light re-emitted from the illuminated area and a spectroscopic analyzer; and an apparatus that measures color based upon absorption of light.

29. The apparatus according to claim 25, including lighting means enabling said region to be illuminated with at least one of visible, ultraviolet, and infrared light.

30. The apparatus according to claim 25, including means enabling colorimetry acquisition to be performed under different kinds of lighting.

31. The apparatus according to claim 25, including means for acquiring relief, said means comprising at least one element selected from the following: a system for projecting fringes; a stereoscopic viewing system; an ultrasound imaging system; and a slit lamp.

32. The apparatus according to claim 25, including means for acquiring mechanical properties, said means comprising at least one element selected from the following: a mechanical feeler; a pinching device; a twisting device; a suction device; a device for projecting a fluid; a tonometer device; and a plethysmographic device.

33. The apparatus according to claim 25, including storage means enabling data acquired at different times to be stored.

34. The apparatus according to claim 25, including a mask arranged to have its entire periphery pressed against a region around the eye.

35. A method of diagnosing at least one state of the region around the eye, the method comprising:
enabling at least one data item to be acquired concerning at least one zone of said region, the data item being associated with at least one of a pigment present in the blood stream and a pigment outside the blood stream;
enabling at least one data item to be acquired relating to at least one of a relief of at least one zone of said region and at least one mechanical property of at least one zone of said region; and
delivering a diagnosis in response to the acquired data.

36. A method of prescribing a treatment for a region around the eye, the method comprising:
enabling at least one data item to be acquired concerning at least one zone of said region, the data item being associated with at least one of a pigment present in the blood stream and a pigment outside the blood stream;
enabling at least one data item to be acquired relating to at least one of a relief of at least one zone of said region and at least one mechanical property of at least one zone of said region; and
prescribing a treatment in response to the acquired data.

37. A kit enabling an individual to perform self-diagnosis for a region around the eye, the kit comprising:
means enabling at least one data item to be acquired concerning at least one zone of said region, the data item being associated with at least one of a pigment present in the blood stream and a pigment outside the blood stream;
means enabling at least one data item to be acquired relating to at least one of a relief of at least one zone of said region and at least one mechanical property of at least one zone of said region; and
means enabling a diagnosis to be delivered at least on the basis of the previously-acquired data.

38. A method of preparing a cosmetic product for a region around the eye, the method comprising:
enabling at least one data item to be acquired concerning at least one zone of said region, the data item being associated with at least one of a pigment present in the blood stream and a pigment outside the blood stream;
enabling acquisition of at least one data item relating to at least one of a relief of at least one zone of said region and at least one mechanical property of at least one zone of said region; and
preparing a product in response to the acquired data.

39. A method of generating a panel of potential users of a cosmetic product for a region around the eye, the method comprising:
for each individual of a group, acquiring:
at least one data item concerning at least one zone of said region, the data item being associated with at least one of a pigment present in the blood stream and
a pigment outside the blood stream;
at least one data item relating to at least one of a relief of at least one zone of said region and at least one mechanical property of at least one zone of said region; and
selecting those individuals of the group for which the acquired data satisfies at least one predetermined criterion.

40. A method according to claim 39, further comprising:
i) generating a panel;
ii) preparing a product;
iii) applying the product to individuals of the panel;
iv) performing a new evaluation:
v) comparing results of the new evaluation with those of at least one preceding evaluation, in order to determine effectiveness of the product; and
vi) if the effectiveness of the product is deemed to be insufficient, modifying at least one of a formulation of the product and a dosage of the product.

41. A method ad recited in claim 40, further including repeating steps iii), iv), and v) until effectiveness is obtained that is deemed to be satisfactory.

42. A method of determining the effectiveness of a treatment for a region around the eye, the method comprising:
performing a first evaluation of the region around the eye by:
(a) enabling acquisition of at least one data item concerning at least one zone of said region, the data item being associated with at least one of a pigment present in the blood stream and a pigment outside the blood stream;\
(b) enabling acquisition of at least one data item relating to at least one of the relief of at least one zone of said region and at least one mechanical property of at least one zone of said region; and
(c) at least on the basis of the previously-acquired data, delivering at least one piece of information relating to the typology of said region;
performing a treatment;
performing a new evaluation; and
comparing results of the evaluations to obtain information concerning effectiveness of the treatment.
